(19) United States
(12) Patent Application Publication
(10) Pub. No.: US 2026/0194490 A1
(43) Pub. Date: Jul. 9, 2026

(54) SYSTEM FOR ADAPTIVE SPECTRAL CALIBRATION

(71) Applicant: IntegenX Inc., Pleasanton, CA (US)

(72) Inventors: Jianbo Gao, Sunnyvale, CA (US); Charles Troup, Livermore, CA (US); Arnaldo Barican, San Ramon, CA (US); Jie Deng, Fremont, CA (US)

(21) Appl. No.: 18/730,264

(22) PCT Filed: Jan. 20, 2023
(86) PCT No.: PCT/US2023/060970

§ 371 Date: Jul. 18, 2024

Related U.S. Application Data
(60) Provisional application No. 63/301,889, filed on Jan. 21, 2022.

(51) Int. Cl. G01N 27/447; C12Q 1/6876

(52) U.S. Cl. CPC G01N 27/44726; C12Q 1/6876; G01N 27/44791; C12Q 2600/166

(57) ABSTRACT

Disclosed are systems and methods for improving the function of analytical instruments used to analyze dye-labelled nucleic acid samples by minimizing spectral anomalies from dye data. A computer system communicatively coupled to the instrument is configured to select multiple different nucleic acids of different sizes and determine dye spectral profiles associated with each of the different nucleic acids. The spectral profiles are used to generate multiple dye matrices each respectively associated with different nucleic acid sizes. When analyzing a test sample, the dye matrices are then separately applied to nucleic acid fragments with sizes similar to the nucleic acids from which the dye matrices were derived, better tailoring the dye matrices to the conditions in which they were generated and minimizing unwanted dye data artifacts.

### CROSS-REFERENCE TO RELATED APPLICATIONS

This application claims priority to U.S. Provisional Application No. 63/301,889 (filed Jan. 21, 2022), titled “SYSTEM FOR ADAPTIVE SPECTRAL CALIBRATION” the entire contents of which are incorporated by reference herein.

### TECHNICAL FIELD

This disclosure relates generally to systems, instruments, and related methods for analyzing dye-labelled samples. Examples include systems, instruments, and related methods for electrophoretic separation and analysis of dye-labelled samples, including capillary electrophoresis applications in which nucleic acid samples are labelled with dyes, size-separated, and analyzed.

### RELATED TECHNOLOGY

The use of fluorescent dyes to label target molecules for detecting, characterizing, or otherwise analyzing the target molecules is ubiquitous. Common applications include the identification and characterization of nucleic acids such as in forensic analysis, human identification, pathogen monitoring, and DNA sequencing.

Often, multiple dyes are used to increase efficiencies by proving parallel channels for analysis. Even though dyes are typically carefully selected to for concurrent use such that each dye of a set peaks at a different wavelength, a given dye's spectral profile usually overlaps with one or more of the other dyes to at least some degree. The particular pattern of overlap between spectral profiles of the different dyes is used to generate a dye matrix to account for the overlap in fluorescence.

A dye matrix compensates for the overlap of different dyes by offsetting, in each dye's detection range, the portion of the overall signal attributable to fluorescence from the other dyes. However, an improperly calibrated dye matrix results in too much or too little compensation of dye spectral overlap, resulting in electropherogram artifacts that can reduce the accuracy of allele typing or other intended analysis.

An improperly calibrated dye matrix can lead to unwanted anomalies in the resulting dye signal data. An example of such anomalies is bleed through peaks or “pull-ups”, which can result from too little subtraction of dye spectral overlap during deconvolution. Pull-ups are problematic because they can lead to incorrect allele calls. For example, pull-ups can make it appear that a particular target nucleic acid was present in the sample when in fact the peak is only an artifact of the improperly calibrated dye matrix.

An elevated interpeak baseline is another artifact in the dye signal data that can occur due to an improperly calibrated dye matrix. This can be the result of too much subtraction of dye spectral overlap during deconvolution. An elevated interpeak baseline is problematic because it can lead to missed allele calls. For example, it can lead to missed detection of a nucleic acid even though it is present in the sample.

Post-hoc computational corrections are sometimes used to correct for observed inconsistencies. However, this type of after-the-fact adjustment can force undesirable and arbitrary anomalies into the dye signal data. It also introduces computational inefficiencies by requiring the use of additional, post-hoc computing to determine and apply the correction factors.

Accordingly, there is an ongoing need for improvements to dye matrix creation and integration with analytical instruments.

### SUMMARY

Described herein are systems and methods for improving the efficiency, accuracy, and/or reliability of procedures that analyze dye-labelled samples. The embodiments described herein are particularly beneficial for improving the efficiency, accuracy, and/or reliability of procedures that analyze dye-labelled samples according to electrophoretic size separation.

In one embodiment, a system is configured to provide spectral calibration for improved analysis of dye-labelled samples by: using a calibration sample having multiple known nucleic acids, determining sizes of two or more different nucleic acids; selecting a first nucleic acid with a first size, and determining a spectral profile associated with the first nucleic acid for a particular dye; using the spectral profile associated with the first nucleic acid, creating a first dye matrix corresponding to the first nucleic acid; selecting a second nucleic acid with a second size, and determining a spectral profile associated with the second nucleic acid for the dye; using the spectral profile associated with the second nucleic acid, creating a second dye matrix corresponding to the second nucleic acid; and incorporating both the first dye matrix and the second dye matrix into a calibration file that can be written into the analytical instrument to enable improved analysis of dye-labelled samples.

The system may further be configured to deconvolute raw fluorescence data of a test sample by determining sizes of two or more different nucleic acids in the test sample and assigning dye matrices to the different nucleic acids based on the determined sizes of the different nucleic acids. For example, the first and second dye matrices may be assigned to respective first and second sets of nucleic acids of the test sample by comparing the sizes of the nucleic acids of the test sample to the sizes of the nucleic acids of the calibration sample from which each dye matrix was generated and mapping the dye matrices to the different nucleic acids of the test sample according to similarities in compared sizes. The system may then output an electropherogram that provides a dye signal plot representing the test sample.

Improved dye signal data generated in this manner beneficially minimizes artifacts and anomalies that can otherwise lead to missed or incorrect allele calls, reduced reliability of results, wasted testing and material resources, and decreased process efficiencies. An improved dye signal plot generated in this manner can also beneficially decrease the time and computing resources that would otherwise be required to account for and correct such anomalies. For example, by reducing the occurrence and/or severity of spectral anomalies, there is a concomitant reduction in the need to expend computing resources for post-hoc correction of obtained spectral data.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates a capillary electrophoresis instrument as an example of one application in which the present systems and methods may be utilized;

FIG. 2 illustrates an exemplary electropherogram as output of an electrophoresis procedure, this example showing a size ladder standard and results of a short tandem repeat (STR) analysis;

FIG. 3A illustrates an exemplary spectral profile for various dyes, showing spectral overlap requiring compensation through the use of a dye matrix;

FIG. 3B illustrates an exemplary spectral profile, prior to spectral separation, for a single scan of a test sample, showing the non-separated, “raw” fluorescence over measured wavelength bins;

FIG. 3C illustrates an exemplary dye signal plot showing separated dye signals for various dyes over time (i.e., over several sequential scans);

FIGS. 4A and 4B illustrates spectral profiles of a SID dye for different alleles and different loci, showing that the spectral profile is affected by the size of the nucleic acid analyzed;

FIGS. 4C and 4D illustrate examples of potential defects that can occur in dye signal plots as a result of a suboptimal dye matrix, with FIG. 4C showing inaccurate “pull-up” peaks and FIG. 4D showing an unwanted elevated interpeak baseline, each of which can be caused by an improperly calibrated dye matrix;

FIG. 4E illustrates an example of a post-hoc adjustment to a dye matrix spectral profile intended to correct for observed inconsistencies, but showing unwanted negative fluorescence values as a result;

FIG. 5 illustrates a data workflow for generating a spectral calibration file that may be utilized to provide effective spectral calibration and improved output in a system for analyzing dye-labelled samples;

FIGS. 6A and 6B illustrate a data workflow for incorporating the spectral calibration file of FIG. 5 into a dye deconvolution process associated with processing of a test sample of unknown nucleic acids in order to generate an improved dye signal plot;

FIG. 7 illustrates an exemplary method for generating a spectral calibration file that may be utilized to provide effective spectral calibration and improved output in an instrument for analyzing dye-labelled samples;

FIG. 8 illustrates an exemplary method for mapping multiple, different dye matrices to different nucleic acids of a test sample in order to generate an improved dye signal plot from an analytical instrument;

FIGS. 9A through 9D illustrate potential unwanted spectral artifacts that can result from Nordman effect residuals in dye data;

FIG. 10 illustrates an exemplary method for generating a Nordman effect correction for use in reducing Nordman residuals in dye data; and

FIG. 11 illustrates an exemplary method for incorporating the Nordman correction into an analytical instrument to generate a corrected dye signal plot.

### DETAILED DESCRIPTION

### Selected List of Defined Terms

As used herein, the terms “raw fluorescence data” and “raw data” refer to fluorescence data prior to spectral separation using a dye matrix or other spectral separation operation. Fluorescence data is thus still considered “raw data” even if subjected to other processing operations so long as it has not yet been spectrally separated. “Raw data” includes fluorescence data gathered over multiple sequential “scans” of a sample, where each scan measures fluorescence over a range of wavelengths (sometimes referred to as discrete wavelength “bins”).

Each scan produces a “spectral profile” of fluorescence as a function of wavelength for that particular scan number. Before spectral separation, a spectral profile indicates the overall fluorescence signal over the measured wavelength range. After spectral separation, the spectral profile is separated into its specific, component dye values.

As used herein, the terms “dye signal data” and “dye signal plot” refer to the fluorescence data, as measured over multiple sequential scans, after spectral separation. After the scans have been spectrally separated into their component dye values, a dye signal data/plot can be generated indicating the separated dye signals over time (i.e., over the multiple sequential scans). The term “electropherogram” may also be used as a synonym for these terms.

### Exemplary Systems & Uses

The systems and methods described herein may be utilized in a variety of applications and with a wide variety of analytical instruments that involve the analysis of dye-labelled samples. Examples of such instruments and related applications include those configured for size separation such as electrophoresis (e.g., capillary electrophoresis), nucleic acid amplification such as polymerase chain reaction (PCR) (e.g., real-time PCR) and loop-mediated isothermal amplification (LAMP), nucleic acid sequencing (e.g., Sanger sequencing, pyrosequencing, or next generation sequencing (NGS)), mass spectrometry, flow cytometry, and spectrophotometry.

The systems and methods described herein may be directed to analysis of nucleic acid molecules, (including DNA and/or RNA), proteins (e.g., enzymes, antigens, antibodies, etc.), lipid molecules, carbohydrate molecules, cellular components, or other biomolecules of interest capable of associating with a dye and/or otherwise capable of emulating a fluorescence signal for analysis.

The present disclosure includes specific examples directed to capillary electrophoresis and analysis of nucleic acid samples using fluorescent dyes. However, the skilled person will understand, in light of this disclosure, that other implementations may additionally or alternatively involve other biomolecules of interest and/or other analytical instruments and applications.

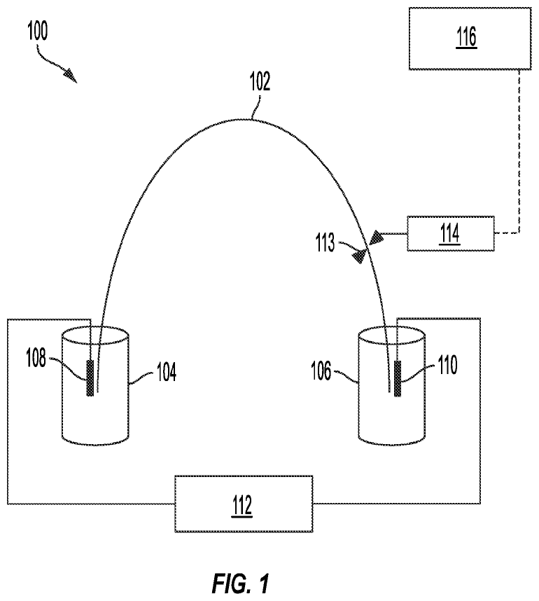

FIG. 1 schematically illustrates a capillary electrophoresis instrument 100 as one example of an application in which the presently described embodiments may be implemented. The capillary electrophoresis instrument 100 includes a capillary 102 with one end in contact with a source container 104 and the opposite end in contact with a destination container 106. The capillary 102 is filled with a sieving polymer. Typically, multiple capillaries are included in a capillary electrophoresis system to allow for parallel analysis of multiple samples, though for the sake of clarity only one capillary 102 is illustrated here. The use of capillaries, as opposed to conventional slab gels, allows for the use of stronger electrical fields, which results in faster separation and increased overall throughput.

The source container 104 and destination container 106 hold an appropriate electrolytic buffer, and the sample to be analyzed is added to the source container 104 or otherwise mixed with the buffer at the source container 104 when introduced into the capillary 102.

The capillary electrophoresis instrument 100 also includes a pair of electrodes 108 and 110 that are in electrical communication with the buffer solution at the opposing containers 104 and 106. A power supply 112 generates a voltage between the electrodes 108 and 110. The sample is introduced into the capillary 102 and the electric potential between electrodes 108 and 110 then causes the targeted analytes to migrate through the capillary 102 toward the destination container 106.

The analytes (e.g., nucleic acids) separate by size as they migrate through the capillary 102 according to differences in electrophoretic mobility through the capillary. That is, smaller fragments will move through the polymer faster than larger fragments. In applications where nucleic acids are the analytical target (as in this example), the negatively charged nucleic acid fragments will move from the negatively charged cathode 108 toward the positively charged anode 110 under the applied voltage.

The capillary 102 includes a detection window 113 coincident with a corresponding detector assembly 114. The detector assembly includes a laser and a fluorescence detector. As dye-labelled nucleic acid fragments pass through the detection window, the laser excites the dye-labelled fragments and the detector (e.g., a charge-coupled device (CCD) camera) detects the resulting fluorescence signals. Typically, multiple different dyes that each provide a different, known fluorescence response to the excitation light are used to label different nucleic acid targets. Thus, the identities of the fragments may be determined according to the character of the corresponding fluorescence signal.

The detector assembly 114 is communicatively coupled to a computer device 116. The computer device 116 includes one or more processors and memory (e.g., one or more hardware storage devices) that enable it to receive the fluorescence signal data from the detector assembly 114 and to generate an electropherogram showing the detected fluorescence signals of the different dye “channels” over time. Peaks in the electropherogram indicate times at which a labelled fragment passed through the detection window 113. By comparing these detected peaks to standard peaks (i.e., a “ladder”) from a sample having fragments of known size, the sizes of the fragments can be determined.

One major use of capillary electrophoresis is STR typing. STR loci are targeted with specified primers and are then amplified and labelled with dyes. Often, multiple dyes are used, each dye being specific to a particular locus or set of loci that are expected to sufficiently spread out once size separated. This allows for allele typing of multiple loci spread among the different dye channels. With enough loci analyzed, the pattern of alleles provides highly accurate identification of an individual. In the U.S., for example, STR typing procedures commonly analyze the standard core loci of the Combined DNA Index System (CODIS), referred to as the CODIS 20 (or the core loci of the previous CODIS 13 standard).

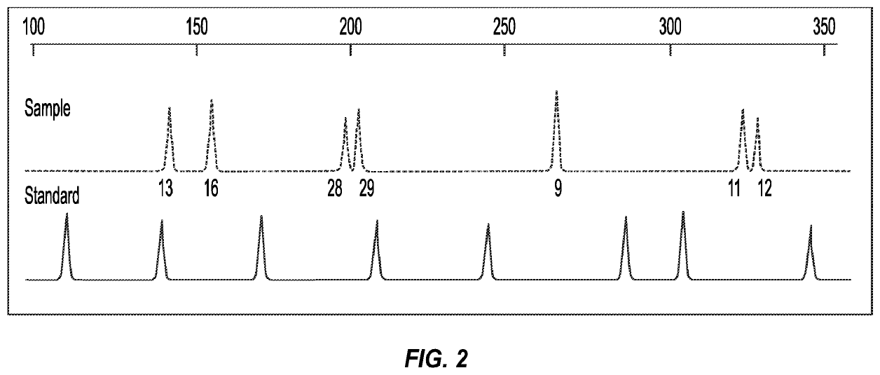

FIG. 2 is an exemplary electropherogram for an STR typing procedure. Common components of such an electropherogram include a size standard showing peaks from nucleic acid fragments of known size, the peaks of the test sample in one or more dye channels (a single channel shown here), and a size indicator showing the corresponding length (in number of base pairs) of the fragments. As shown, the peaks of the test sample are usually also annotated with a number to indicate the STR allele(s) detected at the particular locus associated with each peak or pair of peaks. Different alleles for a given STR locus have different sizes due to different numbers of the repeat sequence present. Loci labels are not shown here but are often also included.

### Dye Matrices & Potential Problems

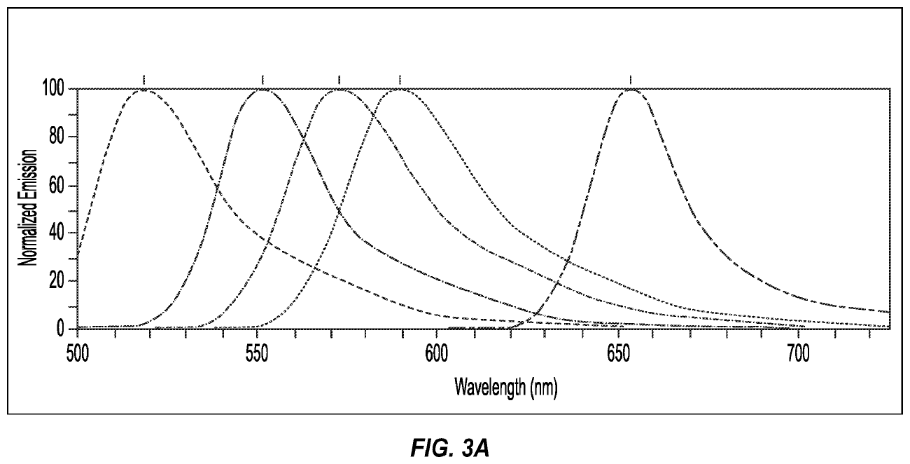

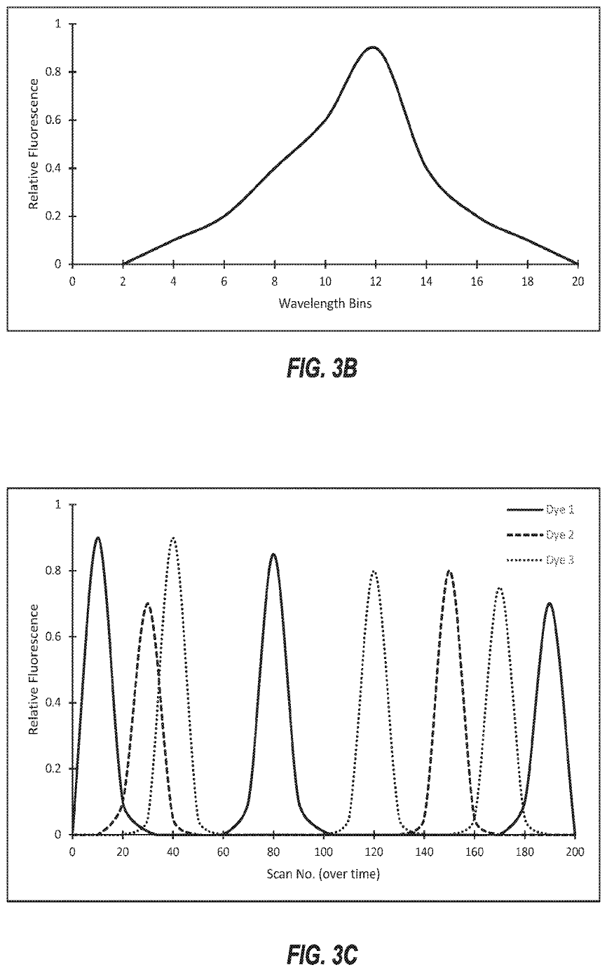

FIG. 3A illustrates an example of a spectral profile for various dyes that may be utilized in an STR application or other application as described above. The spectral profile shows the relative fluorescence of each dye (vertical axis) over a range of different wavelengths (horizontal axis). As shown, even though each dye peaks at a different wavelength, a given dye's spectral profile typically overlaps with one or more dyes to at least some degree. As is known in the art, the particular pattern of overlap between spectral profiles of the different dyes is used to generate a dye matrix to compensate for the overlap in fluorescence. A dye matrix compensates for the overlap of different dyes by offsetting, in each dye's detection range, the portion of the overall signal attributable to fluorescence from the other dyes. However, as described in more detail below, an improperly calibrated dye matrix results in too much or too little compensation of dye spectral overlap, resulting in electropherogram artifacts that can reduce the accuracy of allele typing or other intended analysis.

In a typical capillary electrophoresis procedure, a series of sequential scans are performed at multiple time points. These scans will vary over time as the analyzed nucleic acid fragments pass through the detection window and the resulting fluorescence signal detected at the detection window changes accordingly. FIG. 3B illustrates an example of the fluorescence data obtained from such a scan, prior to spectral separation of the fluorescence signal. As shown, the scan provides the fluorescence signal over a range of wavelengths and/or series of wavelength bins. Applying the dye matrix to these scans separates the overall fluorescence signal into its component values specific to each dye, and thereby allows the generation of a dye signal plot as shown in FIG. 3C. The dye signal plot illustrates the separated dye signals over time (i.e., over the sequential scan numbers). Although three separate dyes are shown in the example of FIG. 3C, additional dyes may be utilized according to particular application needs and/or preferences. For example, some embodiments may concurrently utilize four, five, or six dyes, or may utilize more than six dyes.

Conventionally, dye matrices are formed assuming that the fluorescence response of any given dye is independent of the particular nucleic acid to which the dye is associated. In other words, dye matrices do not conventionally assume that a spectral profile for a given dye will differ based on the size of the nucleic acids it associates with. However, the present inventors have discovered that, at least in some circumstances, dye spectral profiles do in fact change depending on the size of the nucleic acid.

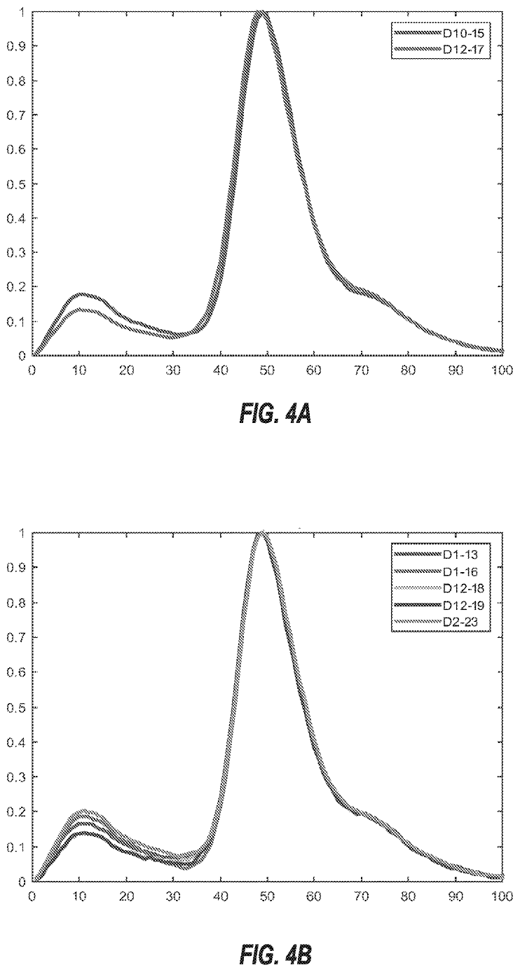

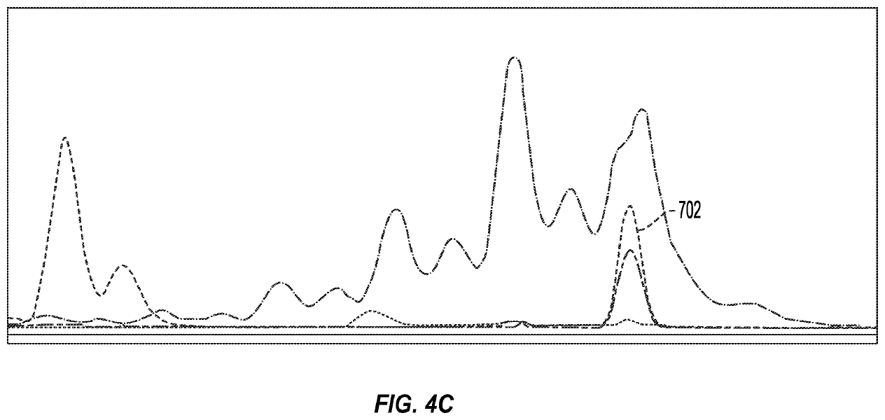

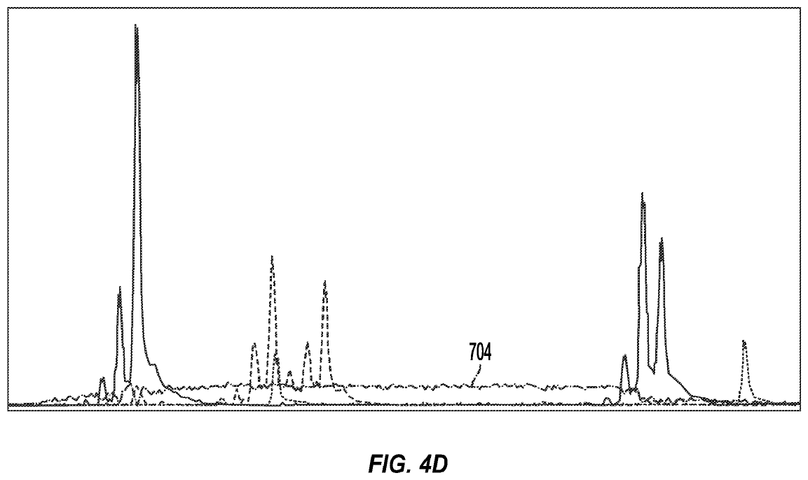

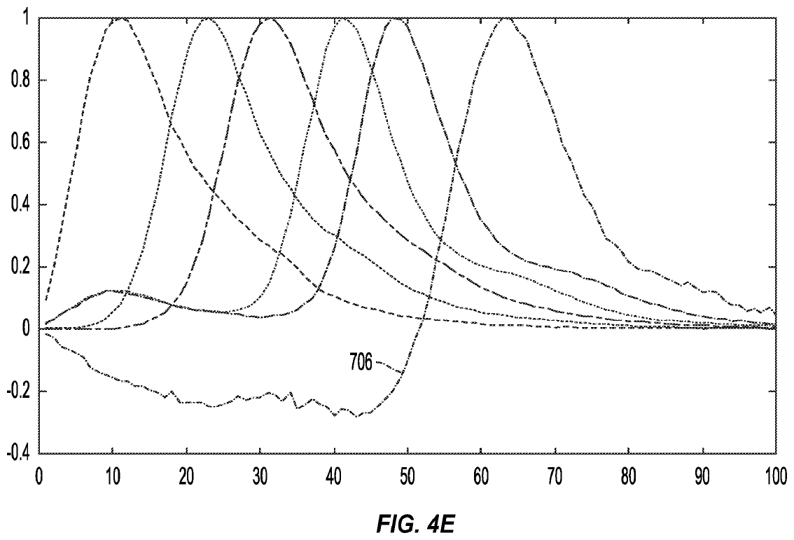

FIG. 4A illustrates spectral profiles for the SID dye at two separate STR loci. As shown, the spectral profiles have apparent differences between the analyzed loci. Similarly, FIG. 4B illustrates spectral profiles for the SID dye at different STR loci and for different alleles for the same locus. FIG. 4B illustrates that not only can different loci result in different spectral profiles, but different allele sizes for the same locus can result in different spectral profile. For example, the profiles for the “D1-13” and “D1-16” alleles have differences, as do the “D12-18” and “D12-19” alleles. Spectra can therefore differ depending on the size of the nucleic acid fragment analyzed.

A dye matrix that does not account for such spectral differences will be suboptimal, particularly for nucleic acid fragments with sizes different from the size(s) used to generate the dye matrix. Similarly, a dye matrix that averages spectral profiles for different nucleic acid sizes will also be suboptimal because certain nucleic acid sizes may provide spectral profiles that differ from the mean. Moreover, setting dye matrix values to change as a function of the measured nucleic acid size (e.g., as a function of the associated scan numbers) is also suboptimal because the observed spectral differences cannot typically be described by an equation that defines dye matrix values as a function of scan number. In other words, the observed spectral differences for different nucleic acid fragment of different sizes result from complex molecular interactions between the nucleic acid fragments and the dye, and an equation (e.g., linear, quadratic, or higher order exponential) is unlikely to adequately capture the resulting dye effects.

An improperly calibrated dye matrix can lead to unwanted anomalies in the resulting dye signal plot. FIG. 4C illustrates an example of bleed through peaks or “pull-ups” 702, which can result from too little subtraction of dye spectral overlap during deconvolution. Pull-ups are problematic because they can lead to incorrect allele calls. For example, pull-ups can make it appear that a particular target nucleic acid was present in the sample when in fact the peak is only an artifact of the improperly calibrated dye matrix.

FIG. 4D illustrates another artifact in the dye signal plot that can occur due to an improperly calibrated dye matrix. Too much subtraction of dye spectral overlap during deconvolution can lead to an elevated interpeak baseline 704. These are problematic because they can lead to missed allele calls. For example, an elevated interpeak baseline 704 can lead to missed detection of a nucleic acid even though it is present in the sample.

FIG. 4E illustrates an example of a dye spectral profile following post-hoc adjustment intended to correct for observed inconsistencies. As shown, however, this type of after-the-fact adjustment can force undesirable and arbitrary anomalies such as negative fluorescent values (see 706). It also requires the use of additional, post-hoc computing to determine and apply the correction factors.

### Adaptive Dye Calibration & Deconvolution

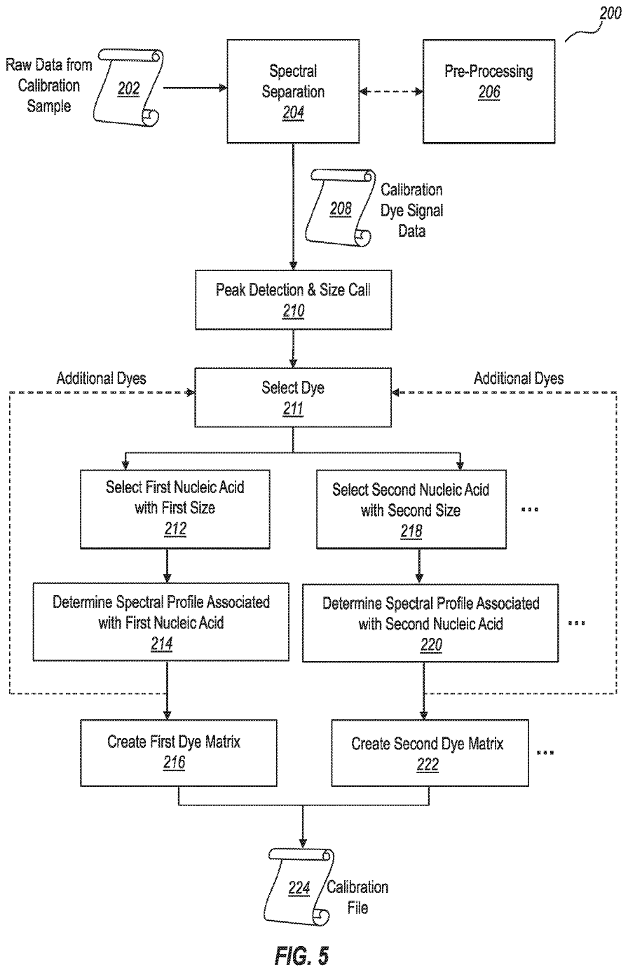

The present embodiments improve upon conventional procedures for forming dye matrices and therefore minimize or avoid the occurrence of unwanted anomalies such as those described above. The data flows and methods shown in FIGS. 5 through 8 may be performed by a computer system (such as computer system 116) that is communicatively coupled to an analytical instrument (such as capillary electrophoresis instrument 100).

FIG. 5 illustrates a data workflow 200 for generating a spectral calibration file that may be utilized to provide effective spectral calibration and improved output accuracy in a system for analyzing dye-labelled samples. In the data flow of FIG. 5, the raw data 202 is generated using a calibration sample that includes multiple dye-labelled nucleic acids of different sizes. The raw data 202 undergoes spectral separation 204 to generate calibration dye signal data 208. The spectral separation of operation 204 may utilize a “default” dye matrix or set of dye matrices. The default dye matrix may be a selected standard default dye matrix, a previously used/calibrated dye matrix or set of dye matrices (e.g., the dye matrix settings from the most recent calibration), or a composite or average dye matrix or set of dye matrices, for example.

The calibration dye signal data 208 can be represented by a dye signal plot such as shown in FIG. 3C, but where the peaks represent known nucleic acids of known sizes.

Optionally, the raw data 202 may also undergo one or more pre-processing operations 206, such as primer trim and chop, adjustment to the dynamic range, baseline correction, band pass filtering, Raman normalization, data rescaling, and/or other data processing operations known in the art. These may be performed prior to and/or after spectral separation 204.

After spectral separation 204, the calibration dye signal data 208 undergoes peak detection and size call operations 210. In these operations, the computer system detects the peaks of the calibration dye signal data and matches the peaks to the corresponding nucleic acid fragment sizes. At this point, the dye data can be utilized to generate multiple (e.g., two or more) dye matrices for use in adaptively calibrating an analytical instrument.

A dye is selected (operation 211). For the selected dye, a first nucleic acid with a first size is selected (operation 212) and a spectral profile associated with the first nucleic acid is determined (operation 214). As shown by the dashed line at the left side of FIG. 5, one or more additional dyes may be selected and the steps of selecting the first nucleic acid with first size (operation 212) and determining a spectral profile associated with the selected nucleic acid (operation 214) are repeated for the one or more additional dyes. The spectral profile(s) associated with the first nucleic acid include data for the selected dyes similar to that shown in FIG. 3A for the various illustrated dyes. This spectral profile data is then utilized to create a first dye matrix (operation 216). Because the first dye matrix was generated from spectral data associated with the first nucleic acid with first size, the first dye matrix is therefore calibrated to nucleic acids of similar size.

Similarly, for each selected dye, a second nucleic acid with second size (different from the first size) is selected (operation 218) and a spectral profile associated with the second nucleic acid is determined (operation 220). As shown by the dashed line at the right side of FIG. 5, one or more additional dyes may be selected and the steps of selecting the second nucleic acid with second size (operation 218) and determining a spectral profile associated with the selected nucleic acid (operation 220) are repeated for the one or more additional dyes. The spectral profile(s) associated with the second nucleic acid include data for the selected dyes similar to that shown in FIG. 3A for the various illustrated dyes. This spectral profile data is then utilized to create a second dye matrix (operation 222). Because the second dye matrix was generated from spectral data associated with the second nucleic acid with second size, the second dye matrix is therefore calibrated to nucleic acids of similar size.

The first and second dye matrices are then combined to form a multi-matrix calibration file 224 that may be utilized to effectively calibrate an analytical instrument in preparation for analyzing test samples of unknown nucleic acids. As shown by the ellipses, some embodiments may include additional dye matrices generated by selecting a third (and optionally fourth, fifth, etc.) nucleic acid of corresponding third (and respective fourth, fifth, etc.) size, and determining a spectral profile associated with the selected nucleic acid for each dye.

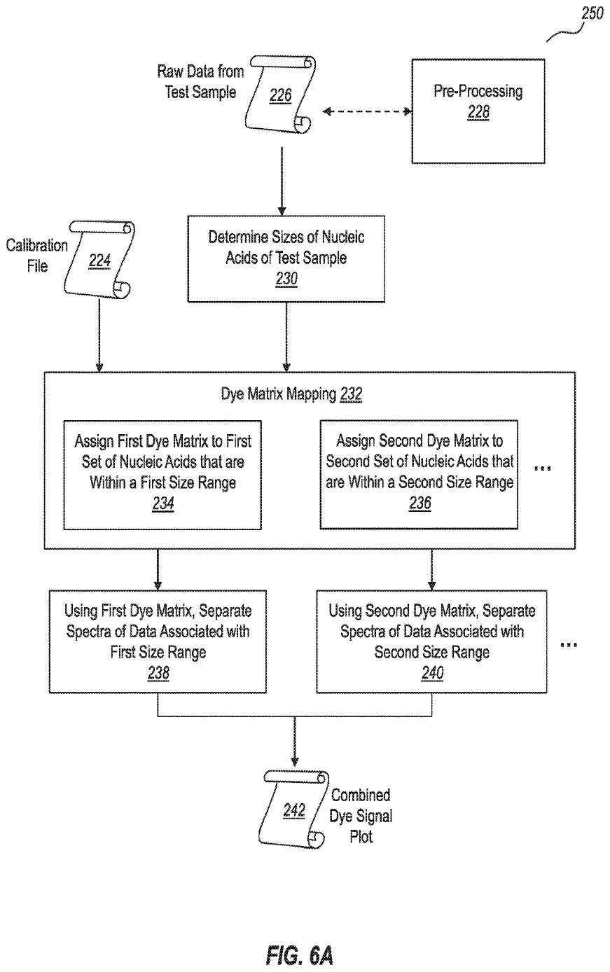

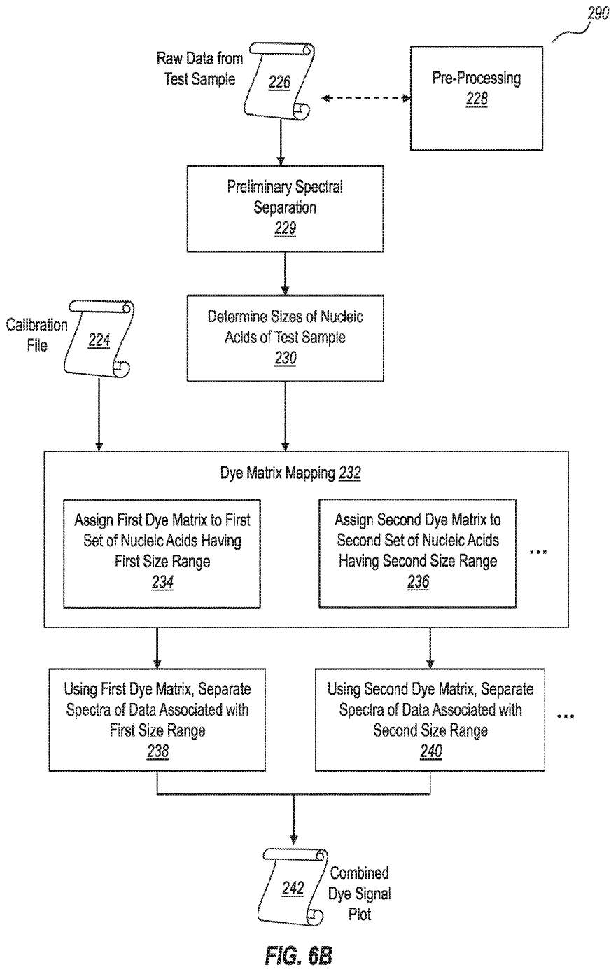

FIG. 6A illustrates a data workflow 250 for incorporating the spectral calibration file 224 of FIG. 5 into a dye deconvolution process. The illustrated workflow may be utilized for analysis of a test sample of unknown nucleic acids in order to generate an improved dye signal plot. As shown, raw data 226 from one or more test samples undergoes a size call operation where the sizes of the nucleic acids of the test sample are determined (operation 230). Optionally, the raw data 226 may additionally undergo one or more pre-processing operations 228 similar to those listed as potential pre-processing operations in the data workflow of FIG. 5.

The sized raw data 226 and the calibration file 224 are then utilized as inputs in a dye matrix mapping operation 232. In the dye matrix mapping operation 232, the computer system assigns the first dye matrix to a first set of nucleic acids that are within a first size range (operation 234) and assigns the second dye matrix to a second set of nucleic acids that are within a second size range (operation 236). The first size range corresponds to the size of the first nucleic acid (selected in operation 212 of FIG. 5) from which the first dye matrix was generated, and the second size range corresponds to the size of the second nucleic acid (selected in operation 218 of FIG. 5) from which the second dye matrix was generated. The ellipses indicate that one or more additional dye matrices may be assigned to additional sets of nucleic acids, each being within a size range that corresponds to the nucleic acid size from which the dye matrix was generated.

The computer system then uses the first dye matrix to separate spectra of data associated with the first size range (operation 238) and uses the second dye matrix to separate spectra of data associated with the second size range (operation 240). The ellipses indicate that one or more additional dye matrices may be used to separate the spectra of data associated with respectively corresponding size ranges. The separated spectra data is then utilized to generate a combined dye signal plot 242.

The use of multiple dye matrices that are each mapped to nucleic acid size ranges from which the respective dye matrices were generated better applies the actual activity of the dye(s) to the spectral separation operation. As a result, the dye signal plot 242 beneficially minimizes anomalies (such as those illustrated in FIGS. 4C-4E) that can otherwise occur when a single dye matrix, an averaged dye matrix, or a dye matrix as a function of scan number is used.

Embodiments described herein can therefore be used to define multiple nucleic acid “size lanes” that are each associated with their own dye matrix that is optimized for that size range. The number of such size lanes and dye matrices to utilize can depend on particular application needs and desired accuracy thresholds. A more granular implementation, with relatively more size lanes and corresponding dye matrices, will provide greater accuracy improvements and will better minimize unwanted anomalies in the dye signal data. On the other hand, even two dye matrices appropriately mapped to their respective size lanes will provides benefits over the conventional approaches and may be sufficient to reach desired accuracy levels.

As an example, an electropherogram for a typical STR application shows a size range from about 50 base pairs to about 450 base pairs in length (though this can vary according to application). The overall size range of roughly 400 may be divided into a number of “lanes”/sections, with the number of such lanes depending on the number of dye matrices utilized. In one example, a first dye matrix may apply to a first size lane covering the range of about 50 to about 250, while a second dye matrix may apply to a second size lane covering the range of about 250 to about 450. These lanes can be more finely divided as more dye matrices are utilized. In some embodiments, the size lanes may be of substantially equal size. Other embodiments may include at least some size lanes of different size. For example, if dye activity is more erratic for one or more dyes at particular subranges, the size lanes may be more granular in those regions to account for the higher size sensitivity.

Embodiments may therefore include two, three, four, five, six, seven, eight, nine, or ten dye matrices each for different corresponding size lanes. Other embodiments may utilize even more than ten dye matrices and size lanes where accuracy, sensitivity, preference, or particular application needs justify it.

FIG. 6B illustrates an alternative embodiment of a data workflow 290 for incorporating the spectral calibration file 224 of FIG. 5 into a dye deconvolution process. The workflow 290 of FIG. 6B is similar to the workflow 250 of FIG. 6A, but includes an additional preliminary spectral separation operation 229 performed prior to the dye matrix mapping operation 232. This preliminary spectral separation operation 229 may be performed to identify the peaks more readily in the separate dye channels of the test sample data and to determine the corresponding alleles/sizes of the peaks. This may be accomplished using a default dye matrix or matrices, as defined above. Once sizes are determined the appropriate dye matrix can be assigned in the subsequent dye matrix mapping operation 232, which will function to minimize suboptimal results of the prior preliminary spectral separation 229.

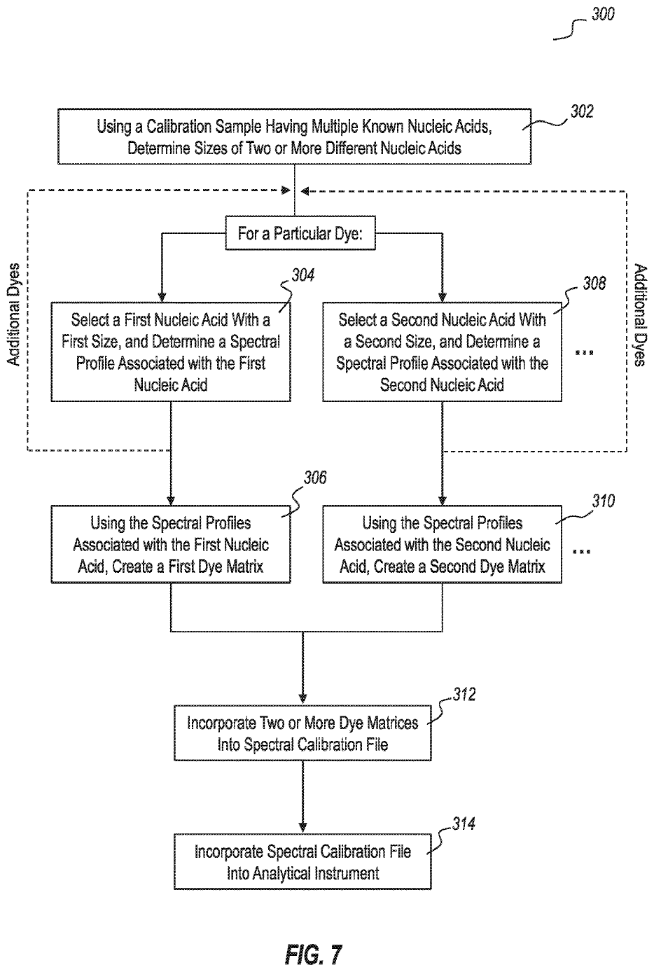

FIG. 7 illustrates an exemplary method 300 for generating a spectral calibration file that may be utilized to provide effective spectral calibration and improved output in a system for analyzing dye-labelled samples. The method 300 can implement the data workflow 200 of FIG. 5, for example, and may be carried out using a computer system communicatively coupled to an analytical instrument such as computer system 116 and instrument 100 shown in FIG. 1.

In the method, the computer system uses a calibration sample having multiple dye labelled, known nucleic acids (e.g., of given sizes) and determines the sizes of two or more different nucleic acids (step 302). For a particular dye, the computer system selects (e.g., according to user input) a first nucleic acid with a first size and determines a spectral profile associated with the first nucleic acid for the particular dye (step 304). This step may be repeated for one or more additional dyes. Then, the computer system uses the determined spectral profiles associated with the first nucleic acid to create a first dye matrix (step 306).

For each of the one or more dyes, the computer system also selects (e.g., according to user input) a second nucleic acid with a first size and determines a spectral profile associated with the second nucleic acid for the dye (step 308). Then, the computer system uses the determined spectral profiles associated with the second nucleic acid to create a second dye matrix (step 310). As indicated by the ellipses, one or more additional nucleic acids with other sizes may also be selected, and the spectral profiles associated with those nucleic acids may be used to create one or more additional dye matrices corresponding to such additional sizes.

The computer system then incorporates the two or more generated dye matrices into a spectral calibration file (step 312). This spectral calibration file may then be written into the analytical instrument (step 314) or otherwise incorporated into the analytical instrument to cause a change in the executable functionality of the analytical instrument.

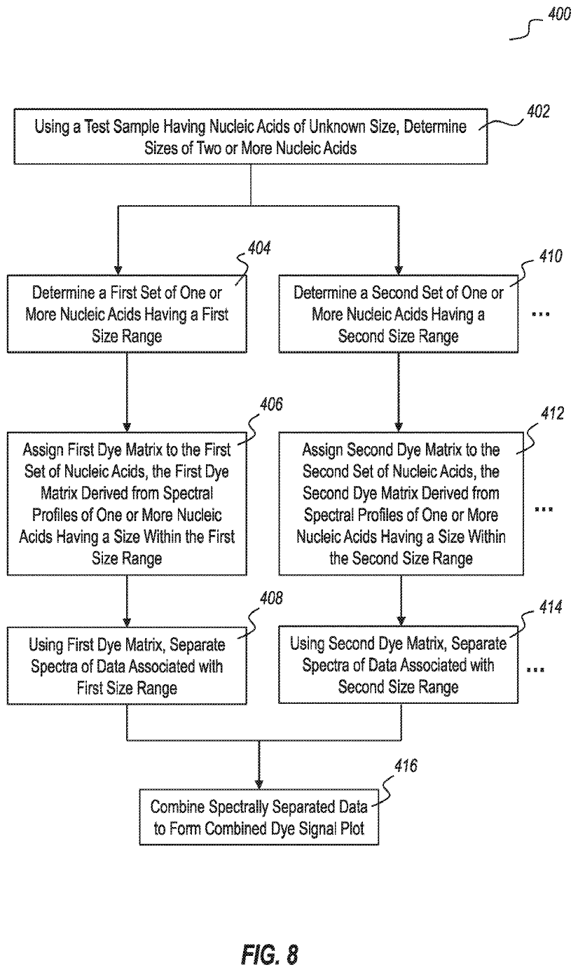

FIG. 8 illustrates a method 400 for mapping multiple dye matrices to different nucleic acids of a test sample in order to generate an improved dye signal plot from an analytical instrument. The method 400 may implement the data workflow 250 and/or 290 of FIGS. 6A and 6B, for example, and may be carried out using a computer system communicatively coupled to an analytical instrument such as computer system 116 and instrument 100 shown in FIG. 1. The method 400 may also be carried out in conjunction with the method 300 of FIG. 7. For example, the calibration file created using the method 300 can be utilized as the source of the dye matrices implemented in the method 400.

In the method 400, the computer system, using a test sample having nucleic acids of unknown size, determines sizes of two or more nucleic acids (step 402). The computer system then determines a first set of one or more nucleic acids having a first size range (step 404) and assigns a first dye matrix to the first set of nucleic acids (step 406). The first dye matrix is derived from spectral profiles of one or more nucleic acids having a size within the first size range. In this manner, the first dye matrix is tailored to the particular nucleic acids within the first size range, and anomalies due to size dependent spectral differences for one or more dyes are minimized. The computer system then uses the first dye matrix to separate spectra of the fluorescence data associated with the first size range (step 408).

The computer system also determines a second set of one or more nucleic acids having a second size range (step 410) and assigns a second dye matrix to the second set of nucleic acids (step 412). The second dye matrix is derived from spectral profiles of one or more nucleic acids having a size within the second size range. The second dye matrix is tailored to the particular nucleic acids within the second size range, and anomalies due to size dependent spectral differences for one or more dyes are thereby minimized. The computer system then uses the second dye matrix to separate spectra of the fluorescence data associated with the second size range (step 414).

The computer system then combines the spectrally separated data from the test sample to form a combined dye signal plot (step 416). Because of the better tailored dye matrices, the resulting dye data beneficially minimizes the undesirable data artifacts that can result from mismatched dye matrices. The ellipses shown in FIG. 8 indicate that one or more additional sets of nucleic acids each associated with other size ranges may also be determined, and corresponding dye matrices derived from nucleic acids having such sizes may be used to spectrally separate the fluorescence data associated with such size ranges.

### Nordman Effect Correction

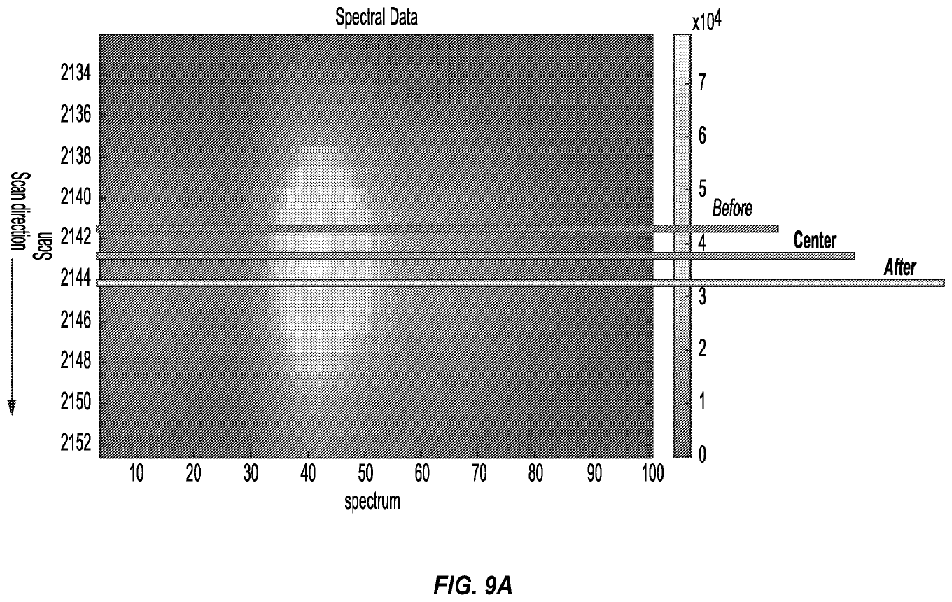

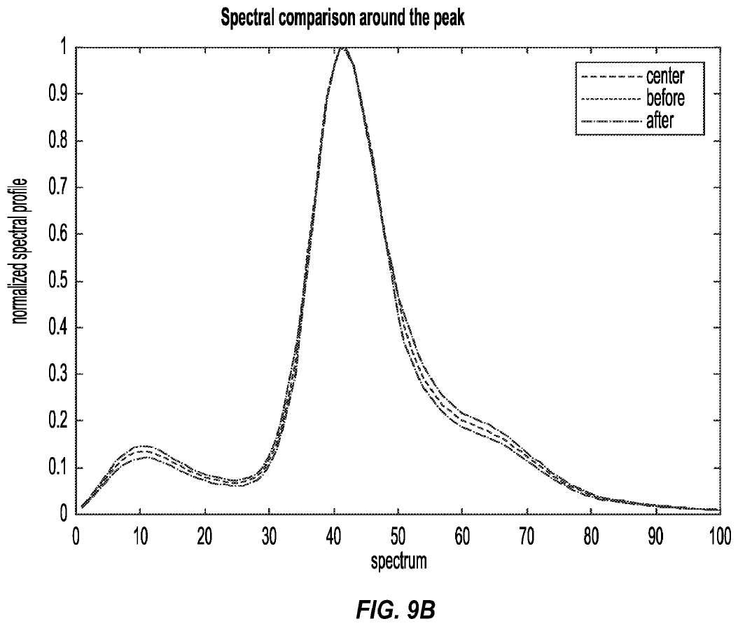

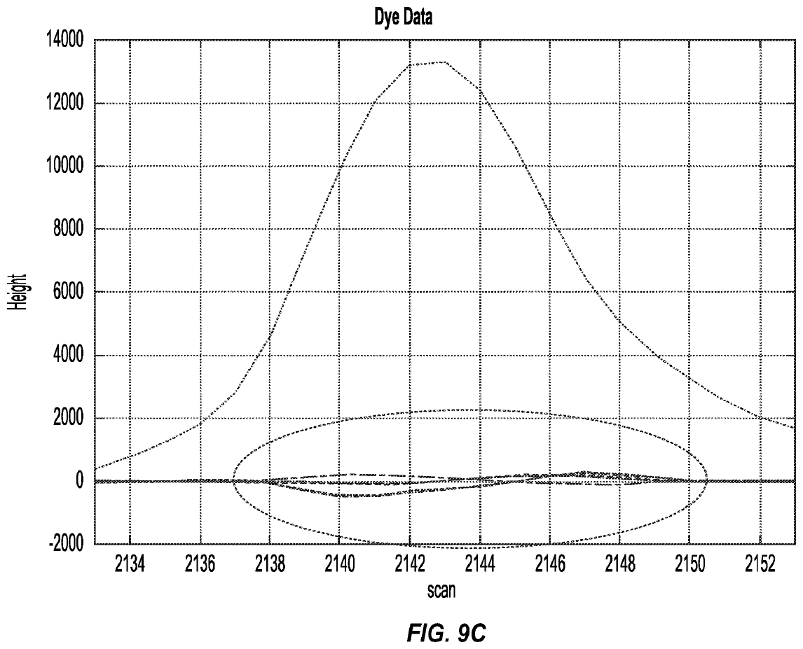

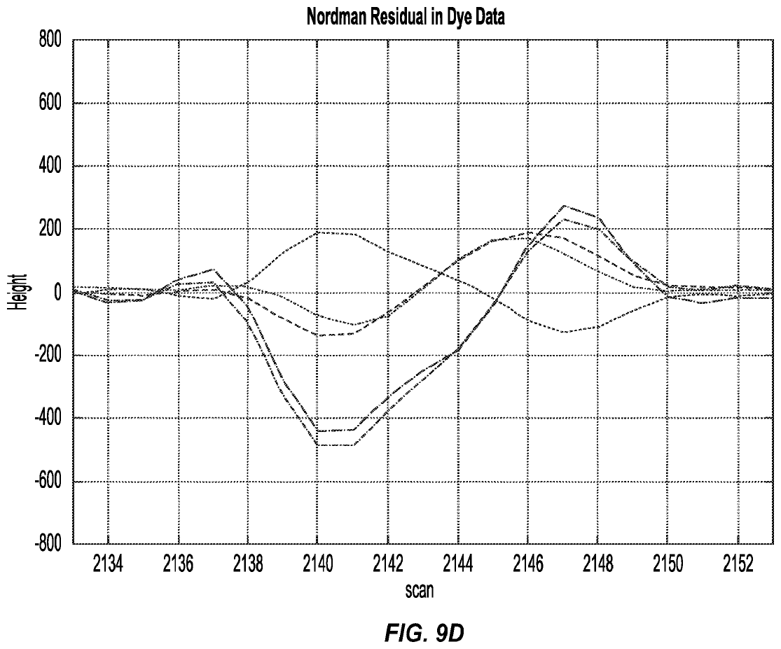

FIGS. 9A through 9D illustrate potential unwanted spectral artifacts that can result from Nordman effect residuals in dye data. FIG. 9A illustrates spectral data for a series of scans for a SID dye, showing a “Before” section generally associated with the scans that occurred slightly before the nucleic acid fragment passed and/or during the initial set of scans during passage of the nucleic acid fragment, a “Center” section generally associated with the scans that occurred during passage of the center of the nucleic acid fragment, and an “After” section generally associated with the later set of scans during passage of the nucleic acid fragment and/or scans that happened slightly after passage of the nucleic acid fragment.

FIG. 9B compares spectral profiles of scans that occurred in the Before, Center, and After sections. As shown, there are differences in the spectral profiles. The “Nordman effect” can cause such spectral differences as a result of when the scan occurred during passage of a nucleic acid fragment. When a dye matrix is generated without accounting for these Nordman effect differences, the resulting dye signal data can inherit unwanted artifacts. FIG. 9C is a dye signal plot illustrating such Nordman residuals/artifacts, and FIG. 9D is an expanded view of the section circled in FIG. 9C to better illustrate the Nordman residuals/artifacts.

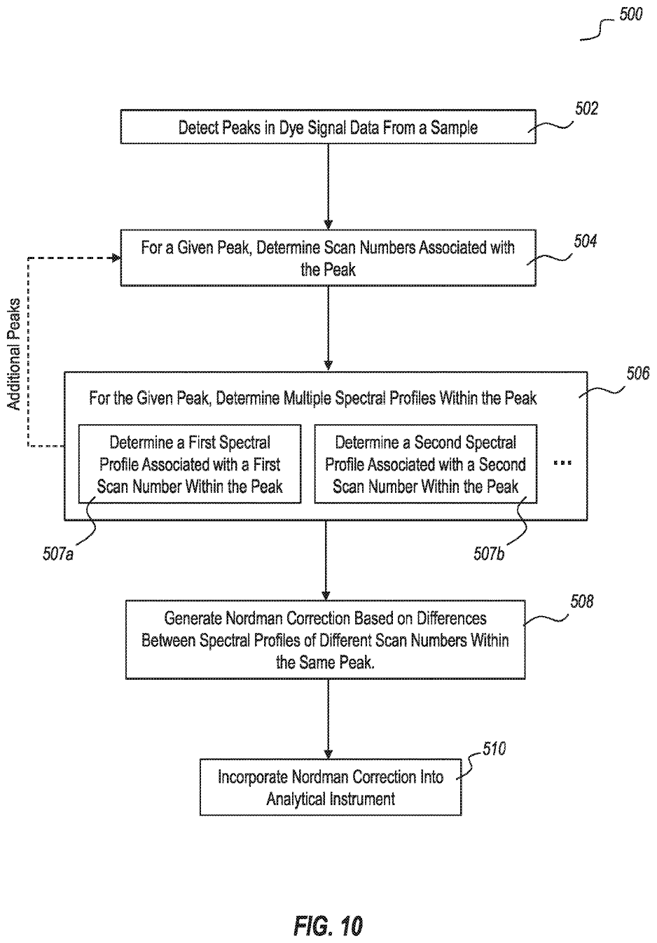

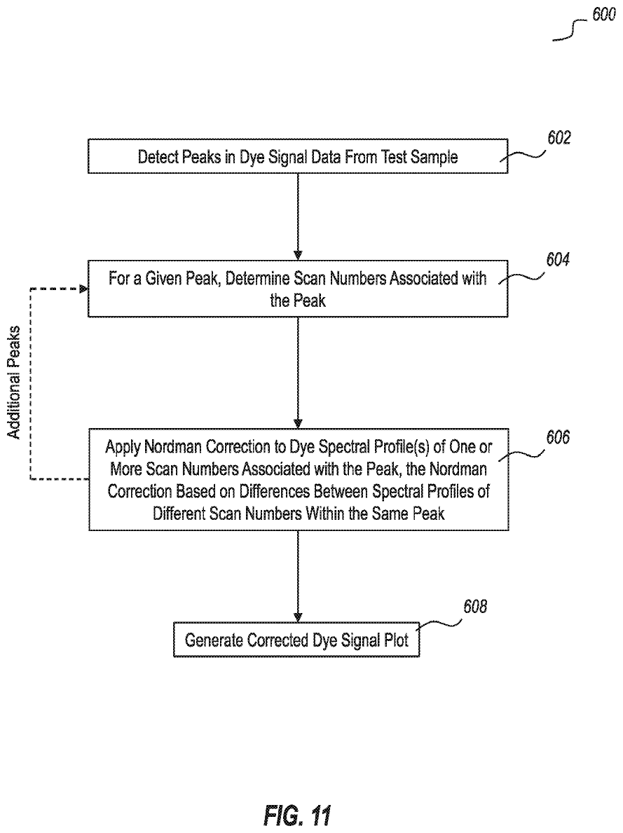

FIG. 10 illustrates an exemplary method 500 for generating a Nordman effect correction for use in reducing Nordman residuals in dye data. Method 500 may be performed by a computer system associated with an analytical instrument, such as computer system 116. Method 500 may be performed independent of methods 300 and/or 400, or in addition to methods 300 and/or 400 to further improve dye signal data obtained from an analytical instrument.

In the method 500, the computer system detects peaks in dye signal data from a sample (step 502). The sample may be a calibration sample with known nucleic acids, for example, though test samples may be utilized in some implementations. For a given peak, the computer system then determines the scan numbers associated with the peak (step 504). For the given peak, the computer system then determines multiple spectral profiles within the peak (step 506). This can include determining a first spectral profile associated with a first scan number within the peak (step 507a), determining a second spectral profile associated with a second scan number within the peak (step 507b), and as indicated by the ellipses, optionally determining one or more additional spectral profiles each associated with a different scan number within the peak.

Because these spectral profiles are associated with different locations of the nucleic acid fragment of the peak, the profiles may differ somewhat because of the Nordman effect. The computer system then generates a Nordman correction based on the differences between the spectral profiles of the different scan numbers within the same peak (step 508). That is, the computer system determines the spectral profile differences as they relate to the scan number (i.e., as they relate to the particular portion within the peak from which they were derived) to generate an appropriate normalizing correction based on the scan number.

Step 506 may be repeated for one or more additional peaks, with the resulting determined spectral profile data also utilized to generate the Nordman correction in step 508. The computer system may then write or otherwise incorporate the Nordman correction into the associated analytical instrument (step 510).

FIG. 11 illustrates an exemplary method 600 for incorporating the Nordman correction into an analytical instrument to generate a corrected dye signal plot. As with other methods described herein, method 600 may be performed using a computer system communicatively coupled to an analytical instrument, such as computer system 116 and instrument 100. Method 600 may be performed in conjunction with method 500 of FIG. 10. For example, the Nordman correction generated in method 500 may be the one used in the method 600.

In the method 600, the computer system detects peaks in the dye signal data from a test sample (step 602). Then, for a given peak, the computer system determines the scan numbers associated with the peak (step 604) and applies the Nordman correction to the dye spectral profile(s) of one or more scan numbers associated with the peak (step 606). The Nordman correction is based on differences between the spectral profiles of different scan numbers within the same peak, and therefore functions to correct spectral differences at different portions of the peak caused by the Nordman effect. The computer system then generates a corrected dye signal plot (step 608) that minimizes Nordman residuals and thus enables more effective analysis of the dye-labelled nucleic acid samples.

### Additional Computer System Details

It will be appreciated that in this description and in the claims, the term “computer system”, “controller”, or “computing system” is defined broadly as including any device or system—or combination thereof—that includes at least one physical and tangible processor and a physical and tangible memory capable of having stored thereon computer-executable instructions that may be executed by a processor. By way of example, not limitation, the term “computer system” or “computing system,” as used herein is intended to include personal computers, desktop computers, laptop computers, tablets, hand-held devices (e.g., mobile telephones, PDAs, pagers), microprocessor-based or programmable consumer electronics, minicomputers, mainframe computers, multi-processor systems, network PCs, distributed computing systems, datacenters, message processors, routers, and switches.

The memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media, which can also be referred to as hardware storage devices.

The computing system also has thereon multiple structures often referred to as an “executable component.” For instance, the memory of computing system can include an executable component for operating the controller and/or functions of the elevation systems and/or circular reciprocation systems disclosed herein. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms—whether expressed with or without a modifying clause—are also intended to be synonymous with the term “executable component” and thus also have a structure that is well understood by those of ordinary skill in the art of computing.

While not all computing systems require a user interface, in some embodiments a computing system includes a user interface for use in communicating information from/to a user. For example, a user interface can be used by a user to dictate their desired operation of the modified magnet assembly. The user interface may include output mechanisms as well as input mechanisms (e.g., I/O Devices). The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product. For the absence of doubt, such computer-readable storage media can also be termed “hardware storage devices,” which are physical storage media—not transmission media.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Those skilled in the art will further appreciate that a computing system may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A system configured to provide spectral calibration for analyzing dye-labelled samples, the system comprising:
one or more processors; and
one or more hardware storage devices having stored thereon computer-executable instructions which are executable by the one or more processors to cause the system to at least:
using a calibration sample having multiple known nucleic acids, determine sizes of two or more different nucleic acids;
select a first nucleic acid with a first size, and determine a spectral profile associated with the first nucleic acid for a particular dye;
using the spectral profile associated with the first nucleic acid, create a first dye matrix corresponding to the first nucleic acid;
select a second nucleic acid with a second size, and determine a spectral profile associated with the second nucleic acid for the dye;
using the spectral profile associated with the second nucleic acid, create a second dye matrix corresponding to the second nucleic acid;
incorporate both the first dye matrix and the second dye matrix into a spectral calibration file; and
write the spectral calibration file into an analytical instrument configured for analyzing dye-labelled samples.

2. The system of claim 1, wherein the system is further configured to pre-process the calibration sample using one or more of the following operations: primer trim and chop; extend dynamic range; correct baseline; band pass filter; Raman normalization; and data rescale.

3. The system of claim 1, wherein the computer-executable instructions further cause the system to use both of the first and second dye matrices to deconvolute raw fluorescence data of a test sample.

4. The system of claim 3, wherein using both of the first and second dye matrices to deconvolute raw fluorescence data of a test sample comprises determining sizes of two or more different nucleic acids in the test sample, and assigning dye matrices to the different nucleic acids based on the determined sizes of the different nucleic acids.

5. The system of claim 4, wherein the first dye matrix is assigned to a first set of nucleic acids and the second dye matrix is assigned to a second set of nucleic acids.

6. The system of claim 5, wherein the first and second dye matrices are assigned to the respective first and second sets of nucleic acids of the test sample by comparing the sizes of the nucleic acids of the test sample to the sizes of the nucleic acids of the calibration sample from which each dye matrix was generated, and mapping the dye matrices to the different nucleic acids of the test sample according to similarities in compared sizes.

7. The system of claim 3, wherein the system is further configured to pre-process the test sample using one or more of the following operations: primer trim and chop; extend dynamic range; correct baseline; band pass filter; Raman normalization; and data rescale.

8. The system of claim 3, the system being further configured to output an electropherogram providing a dye signal plot representative of the test sample.

9. The system of claim 1, wherein the system is an electrophoresis system.

10. The system of claim 9, wherein the system is a capillary electrophoresis system.

11. The system of claim 1, wherein the system is a polymerase chain reaction (PCR) system.

12. The system of claim 11, wherein the system is a real-time PCR system.

13. The system of claim 1, wherein the system is a nucleic acid sequencing system.

14. The system of claim 1, wherein the system is further configured to:
select one or more additional nucleic acids in addition to the first and second nucleic acids;
determine spectral profiles associated with each of the one or more additional nucleic acids;
using the spectral profiles associated with the one or more additional nucleic acids, create one or more additional dye matrices corresponding to the one or more additional nucleic acids; and
incorporate the one or more additional dye matrices into the calibration file.

15. The system of claim 1, wherein the dye is FAM, VIC, NED, SID, TAZ, LIZ, JUN, ABY, or an Alexa Fluor label.

16. The system of claim 15, wherein the dye is SID.

17. The system of claim 1, wherein the system is further configured to, for each of one or more additional dyes:
select two or more nucleic acids of the calibration sample of different size;
determine spectral profiles respectively associated with each of the two or more nucleic acids;
use the spectral profiles associated with the two or more nucleic acids to modify one or both of the first dye matrix or second dye matrix, or create one or more additional dye matrices.

18. The system of claim 17, wherein the system is configured to modify the first dye matrix where one of the two or more nucleic acids matches or has a size substantially similar to the first nucleic acid.

19. The system of claim 17, wherein the system is configured to modify the second dye matrix where one of the two or more nucleic acids matches or has a size substantially similar to the second nucleic acid.

20. The system of claim 17, wherein the system is configured to create one or more additional dye matrices where at least one of the two or more nucleic acids has a size different than the first nucleic acid and different than the second nucleic acid.

21. The system of claim 1, wherein the system is further configured to:
detect one or more peaks in a dye signal plot based on the calibration sample;
for a given peak, determine scan numbers of the dye signal plot associated with the peak;
determine two or more spectral profiles, each associated with a different scan number of the peak;
based on differences between the spectral profiles of the different scan numbers within the peak, generate a Nordman correction for use in further calibrating the system to enable analysis of dye-labelled samples.

22. The system of claim 21, wherein the system is further configured to:
detect one or more peaks in a dye signal plot based on a test sample;
for a given peak, determine scan numbers of the dye signal plot associated with the peak; and
at one or more scan numbers associated with the peak, apply the Nordman correction to dye values associated with the scan numbers.

23-41. (canceled)
